(19) United States
(12) Patent Application Publication
(10) Pub. No.: US 2014/0276150 A1
(43) Pub. Date: Sep. 18, 2014

(54) Apparatus for Acoustic Measurements of Physiological Signals with Automated Interface Controls

(71) Applicants: Ying Sun, West Warwick, RI (US); Brittany Alphonse, Shrewsbury, MA (US); Andrew Spiewak, Southborough, MA (US); Erik Walder, Essex, MA (US)

(72) Inventors: Ying Sun, West Warwick, RI (US); Brittany Alphonse, Shrewsbury, MA (US); Andrew Spiewak, Southborough, MA (US); Erik Walder, Essex, MA (US)

(21) Appl. No.: 14/202,900

(22) Filed: Mar. 10, 2014

Related U.S. Application Data
(60) Provisional application No. 61/792,311, filed on Mar. 15, 2013.

(51) Int. Cl. A61B 7/04; A61B 5/00; A61B 5/08; A61B 5/024; A61B 5/026

(52) U.S. Cl. CPC A61B 7/04; A61B 5/024; A61B 5/026; A61B 5/08; A61B 5/6822; A61B 5/6823; A61B 5/6831; A61B 5/42; A61B 5/4528
USPC 600/504; 600/586; 600/528; 600/529

(57) ABSTRACT

This invention is concerned with a method and apparatus for measuring and controlling the quality of physiological acoustic signals, which include tracheal breathing sounds, lung sounds, heart sounds, blood flow sounds, joint sounds, and gastrointestinal sounds. The interface between the skin and the device is carefully controlled to achieve a desirable acoustic coupling. A pneumatic feedback control system automatically adjusts of the pressure applied to the skin; another pneumatic control system adjusts the pressure inside an airtight chamber for housing the acoustic sensor. A processor assesses the signal qualities, such as amplitude and frequency spectrum, and provides feedback controls to the interface if needed. The resulting method and apparatus eliminates operator's variability and acquires physiological acoustic signals with consistent and desirable qualities for various medical diagnostic purposes.

### FIELD OF INVENTION

This invention relates to a method, system and apparatus that automatically controls the quality of measured body sounds for quantitative analyses of human physiology and diseased conditions.

BACKGROUND OF INVENTION

This invention is concerned with a method and apparatus for measuring physiological acoustic signals with an automated pressure control system for the device-skin interface. The motivation of this invention came from a previous study involving the use of acoustic signals from a stethoscope for identifying individuals at risk of obstructive sleep apnea (OSA). While it was possible to develop signal processing methods to extract parameters for detecting OSA, the stethoscope-skin interface was a major determinant for the quality of the measured acoustic signals. A study involving 30 subjects was approved by the Institutional Review Board (IRB) of the University of Rhode Island to assess the effect of varying the stethoscope-skin interface on the frequency spectrum of the breathing sound. The results showed that the frequency spectrum changed significantly under different applied pressures and with the presence of a double-sided stethoscope adhesive tape (Spiewak et al.).

Although the stethoscope is a ubiquitous tool for medical diagnostics for many decades, it has mainly been used for qualitative, not quantitative, purposes. When applied to the patient, the stethoscope probe is usually held using a hand by a medical professional. A small change in the applied pressure can alter the frequency spectrum of the recorded acoustic signal in a significant way, which is often undetectable by the human ears. In 1965, Howell and Aldridge recognized the effect of stethoscope-applied pressure on the frequency spectrum of the measured sound; they also proposed a modification of the stethoscope diaphragm to improve the discrimination of frequencies. However, then- design still relies on the manually applied pressure, which introduces the operator's variability. To eliminate the operator's variability, a double-sided adhesive tape can be used to attach the stethoscope probe to the patient. However, our study has shown that the double-sided tape reduces the signal level by at least an order of magnitude and affects the frequency spectrum of the measured sound (Spiewak et al.).

Another study was conducted to develop a handle for the stethoscope probe with an embedded force sensor (Alphonse et al.). Because the insertion of the force sensor directly at the probe-skin interface would block the transfer of acoustic signals, the sensor is placed between the handle and the stethoscope probe. Assuming equilibrium of pressure transfer, the sensor-embedded handle can provide an indirect measurement of the applied pressure at the probe-skin interface.

### BRIEF DESCRIPTION OF THE DRAWINGS

The following description may be further understood with reference to the accompanying drawings in which:

FIG. 1 shows an illustrative sectional view of an acoustic measurement device in accordance with an embodiment of the invention;

FIG. 2 shows an illustrative sectional view of an acoustic measurement device in accordance with another embodiment of the invention;

FIG. 3 shows an illustrative diagrammatic view of a system for measuring breathing sound from a throat area of a subject in accordance with an embodiment of the invention;

FIGS. 4A-4C show illustrative diagrammatic views of further systems for measuring acoustic information from a subject in the areas of a chest, an arm and a leg in accordance with further embodiments of the invention;

FIG. 5 shows an illustrative diagrammatic view of a computation and feedback control system in accordance with an embodiment of the invention; and

FIG. 6 shows an illustrative diagrammatic view of a computation and feedback control system in accordance with a further embodiment of the invention

The drawings are shown for illustrative purposes only.

### DETAILED DESCRIPTION

The invention includes an acoustic measurement device attached to the human body with a fabric strap and a hook and loop fastener. The acoustic signal is measured by a probe that has a microphone in an airtight chamber with a diaphragm in contact with the skin. The applied pressure is controlled by an inflatable bladder and a force sensor positioned between the probe and the fabric strap. The inflatable bladder can be inflated or deflated by a pneumatic pump under the control of a processor. Another pneumatic pump controls the pressure in the airtight chamber, which affects the acoustic coupling between the diaphragm and the microphone.

FIG. 1 shows an embodiment of this invention. The probe consists of an airtight chamber (1) that has a diaphragm (2) in contact with the skin. An acoustic sensor such as a microphone (3) is used to pick up sound vibrations from the diaphragm. A thin flat force sensor (4) measures the force applied to the probe from an inflatable rubber bladder (6) attached to the probe via a spring loaded mechanism (5). The inflatable bladder and the probe is secured to the human body with a fabric strap (7). Similar to an inflatable cuff of a sphygmomanometer for blood pressure measurement, the fabric strap is fastened snugly to the body with the bladder deflated. Then, the bladder is inflated or deflated via a connector (9) to achieve the desirable applied force (10) on the force sensor. This force can be calibrated to the pressure (11) at the probe-skin interface. The pressure (12) in the airtight chamber exerted on the diaphragm from inside is controlled by another pneumatic pump via the connector (8).

The flat diaphragm (2) can be replaced by a concave one (13) to conform with the shape of the body better. It can also be flipped over in a convex configuration (14). In other words, with two replaceable diaphragms it is possible to have three different configurations: flat, concave, and convex. This design of a concave/convex diaphragm is different from that proposed by Howell and Aldridge in both its shape and its purpose. The diaphragm proposed by Howell and Aldridge is slightly bowed and has a small raised area in the center of the diaphragm to magnify even further the increased tension resulting from pressure against the skin. The concave/convex diaphragm in the present invention provides a better conformation with the skin surfaces for certain parts of the body; it is not intended to affect the applied pressure. The pneumatic system controls both the skin-probe pressure on the outside of the diaphragm and the chamber pressure on the side of the diaphragm.

FIG. 2 shows an alternative design for sensing the applied pressure. The inflatable rubber bladder (6) is driven by airflows (17) from a pneumatic pump (not shown) via a connector (9). Instead of using a force sensor (element 4 in FIG. 1), a pneumatic pressure sensor (18) is used to measure the bladder pressure. At equilibrium of pressure transfer the bladder pressure should be directly related to the force (10) applied to the probe-skin interface.

FIG. 3 shows a schematic diagram for measuring the breathing sound from the throat area. The probe with a convex diaphragm (20) is positioned at the suprasternal notch just below the laryngeal prominence, and secured with the fabric strap (7). In addition to the probe the hardware system consists of the chamber pressure pneumatic control unit (30), the applied pressure pneumatic control unit (40), the acoustic signal amplifier (50), and the processor (60). The chamber pressure pneumatic control unit (30) contains an air pump and a pressure sensor. It sets the pressure in the airtight chamber via a hose (32) to achieve a desirable coupling between the diaphragm and the microphone. The connection (31) is bidirectional: The chamber pressure pneumatic control (30) sends the chamber pressure signal to the processor (60) and receives a feedback signal to increase or decrease the chamber pressure. The applied pressure pneumatic control unit (40) contains an air pump and an amplifier for the force sensor signal that comes from the probe (20) via line (42). The applied pressure is regulated via a hose (41). The connection (43) is bidirectional: The applied pressure pneumatic control (40) sends the force sensor signal to the processor (60) and receives a feedback signal to increase or decrease the applied pressure. The acoustic signal amplifier (50) receives the signal from the microphone via line (51). After amplification the analog acoustic signal is sent to the processor (60) for digitization and further processing.

FIGS. 4A-4C show other parts of the human body to which the proposed system can be applied. The system can be strapped to the chest (65) to measure the heart sounds or the lung sounds, the abdomen (66) to measure the gastrointestinal sounds as shown in FIG. 4A, over a blood vessel (67) to measure the blood flow sounds as shown in FIG. 4B, or near a joint (68) to measure the joint sounds as shown in FIG. 4C. For each measurement site, the hardware and software of the system remain the same in general. The only things that may need to be changed/adjusted are the diaphragm and the length of the strap.

Computational and feedback control algorithms are implemented in the processor (60). FIG. 5 shows one possible way to control the probe-skin interface by direct setting of the desirable applied pressure and chamber pressure. The Direct Feedback Control (70) can be a simple negative feedback algorithm by comparing the difference between the measured pressures and the desired pressures. FIG. 6 shows a more sophisticated control method that uses the measured acoustic signal as the input. Features such as the frequency spectrum or fractal dimensions are extracted from the acoustic signal (80). These features are used as input to feedback control algorithms (81). Then the computed applied pressure and chamber pressure are inputted to the Direction Feedback Control (70), which is the same as that in FIG. 5.

1. An apparatus for measuring a physiological acoustic signal from human body with an automated interface control, comprising:
a. a probe that is secured to a part of the human body with an adjustable strap;
b. an inflatable air bladder between the strap and the probe for adjusting the applied pressure at the probe-skin interface;
c. a pneumatic control unit to adjust the pressure in the inflatable air bladder;
d. an acoustic sensor in an airtight chamber of the probe;
e. a pneumatic control unit to adjust the pressure in the airtight chamber;
f. a diaphragm in the airtight chamber that contacts the skin;
g. an acoustic signal amplifier that sends the acoustic signal to a processor for computation; and
h. feedback control algorithms implemented in the processor to achieve the desirable applied pressure and the chamber pressure.

2. The apparatus of claim 1, wherein the physiological acoustic signal is selected from a group consisting of tracheal breathing sound, lung sound, heart sound, blood flow sound, joint sound, and gastrointestinal sound.

3. The apparatus of claim 1, wherein the part of the human body is selected from a group consisting of throat, chest, abdomen, blood vessels, and joints.

4. The apparatus of claim 1, wherein the airtight chamber contains a replaceable diaphragm contacting the skin.

5. The apparatus of claim 1, wherein the applied pressure is measured by use of a force sensor between the bladder and the airtight chamber.

6. The apparatus of claim 1, wherein the applied pressure is measured by use of a pneumatic sensor for the bladder pressure.

7. The apparatus of claim 1, wherein the diaphragm is flat, concave, or convex for suitable conformation with the body surface.

8. The apparatus of claim 1, wherein the applied pressure control algorithm employs direct pressure feedback using frequency spectrum or a fractal dimension of the measured acoustic signal.

9. A method for measuring a physiological acoustic signal from human body with automated interface controls, comprising:
a. securing a probe containing an airtight chamber to a skin of the human body with an adjustable strap;
b. adjusting the applied pressure in the airtight chamber using a pneumatic control unit between the strap and the probe;
c. detecting a acoustic signal using an acoustic sensor in an airtight chamber of the probe;
d. sending the acoustic signal to an acoustic signal amplifier;
e. transmitting the amplifier signal to a processor for computation; and
f. using a feedback control algorithms in the processor to achieve the desirable applied pressure and the chamber pressure.

10. The method of claim 9, wherein the physiological acoustic signal is selected from a group consisting of tracheal breathing sound, lung sound, heart sound, blood flow sound, joint sound, and gastrointestinal sound.
